(19) United States
(12) Patent Application Publication
(10) Pub. No.: US 2005/0228324 A1
(43) Pub. Date: Oct. 13, 2005

(54) Machine and process for lining and/or cushioning orthopedic casts and other orthopedic devices

(76) Inventor: John Louis Stanton, Clarkville, TN (US)

Correspondence Address: DAVID DOUGLAS WINTERS, SUITE 237, 2277-C WILMA RUDOLPH BLVD, CLARKSVILLE, TN, 37043-5898, US

(21) Appl. No.: 11/063,009

(22) Filed: Feb. 22, 2005

Related U.S. Application Data
(63) Continuation-in-part of application No. 10/819,650, filed on Apr. 7, 2004.

(51) Int. Cl. A61F 5/00

(52) U.S. Cl. 602/8

(57) ABSTRACT

A device and process comprised of or employing hydrophobic synthetic polymers used for cushioning against and/or protection from from chaffing, pressure point injury, damage or discomfort from an orthopedic or non-orthopedic, cast, compression dressing, and/or brace, and to protect against a cast saw blade.

### CROSS REFERENCE TO RELATED APPLICATIONS

This is a continuation-in-part of application Ser. No. 10/819,650, filed 07 Apr. 2004.

### STATEMENT REGARDING FEDERALLY SPONSORED RESEARCH OR DEVELOPMENT

Not Applicable

### DESCRIPTION OF ATTACHED APPENDIX

Not Applicable

Lexicon

Use of the word “impervious” in this patent is intended to mean “not affected by.”

For the purposes of this patent, a “brace” is defined as “an orthopedic appliance that supports or holds in correct position, any movable member or part of the body, and that allows motion of that member or part, in contrast to a splint, which prevents motion of the part.”

For the purposes of this patent, a “cast” is defined as “a rigid encasement of a member or part to immobilize that member or part, in contrast to a splint, which immobilizes, but does not encase, the member or part

### BACKGROUND OF THE INVENTION

1. This invention relates generally to the field of orthotic, or orthopedic device liners and more specifically to a machine and process for lining and/or cushioning orthopedic casts and other orthopedic devices.

2. The method presented in this patent has been exercised on volunteer human subjects with unqualified success. Properly administered, it provides for significantly improved comfort, freedom of activities and freedom from inflammation or infection over previously known technologies.

3. The following patents and product literature are representative of the previously known art in the field.

4. U.S. Pat. No. 4,194,041 (Gore, et al) describes a waterproof and breathable polytetrafluoroethylene (hereinafter PTFE) laminate which allows high water vapor transmission even under adverse climatic conditions. Such laminates in modified forms are commercially available from W. L. Gore & Associates. The waterproof and breathable laminate described in this patent consists of a flexible outer layer of micro porous hydrophobic material, such as porous, expanded PTFE, (hereinafter ePTFE) and a second interior layer of continuous hydrophilic elastomeric material attached to the interface of the outer layer. The interior layer does not permit passage of liquid water. The material is specified for use in the fabrication of waterproof, breathable garments, tents, and the like.

5. U.S. Pat. No. 4,989,593 (Campagna, et al) discloses a rigid orthopedic cast or splint which includes padding treated with a fluorochemical or silicone. The padding is able to shed water rapidly promoting comfort for the user.

6. U.S. Pat. No. 5,016,622, (Norvell) discloses an orthopedic cast and padding assembly having a water impermeable, water vapor permeable membrane in contact with the skin, a resilient padding layer of conventional material, and a plaster or resin/glass fiber outer immobilizing layer. The assembly is said to increase the comfort of a wearer and reduce bacteria contained within the cast.

7. Another cast material is found in U.S. Pat. No. 5,102,711 (Keller, et al). This patent discloses a flexible, breathable, composite having a middle layer of conventional man-made or natural fiber padding and a top and bottom layer comprising a sheet of water impermeable, water vapor permeable film bonded to the middle layer. Preferably the top and bottom layers comprising (a) a flexible first sheet of hydrophobic material; and (b) a continuous hydrophilic sheet attached to or penetrating the first sheet forming a barrier to passage of fluids. The composite material is useful in applications such as liners under orthopedic casts and as thermal insulation in apparel.

8. U.S. Pat. No. 5,397,628, (Crawley, et al) discloses a body protection material having an inner layer of ePTFE laminated to an outer layer of substantially air impermeable cellular rubber.

9. There exists a need, unfilled by the above patents, and primarily with respect to orthotic or orthopedic purposes, for a means of providing, and an orthopedic system using, effective, inexpensive, fast drying, non-allergenic, waterproof protection and padding, that can be used in direct contact with the skin of a wearer. Applications include various garments such as sleeves, socks and gloves, liners for orthotic devices such as casts and braces and orthotic devices using such liner/padding material.

10. Cast and brace liner applications are exemplary of the need for waterproof fabrics and of the lack of suitable fabrics in the prior art to fulfill these needs. Various orthopedic and orthotic devices and processes are commonly used to reinforce or immobilize a portion of the body of the wearer, usually a limb or joint. Casts are commonly employed when treating a bone fracture.

11. In applying a conventional cast; it is common practice to first wrap or otherwise cover the body member being treated with a protective fabric made of a material such as cotton or polyester knit. The thus protected body member is then covered with padding, also of cotton or polyester, to provide a soft, resilient cushion/lining.

12. The padding is then over-wrapped with a cast of plaster and woven cloth, or of fiberglass material in the form of heat-softened or curable organic polymer resins, coated on glass fibers formed into fabrics, which are wrapped about the body member and thereafter hardened by cooling or by application of specific energy such as ultra-violet radiation.

13. Similarly, art is also known for applying various braces and orthotic devices to the body of a wearer, mainly to the limbs and joints. In some applications, these braces are compression members. Others may incorporate some type of range of motion limiting device. Such devices are widely used in therapeutic applications and are also widely used in connection with athletic activities, particularly to protect knee joins from injury.

14. Use of orthopedic casts, orthotic devices and braces often causes discomfort to the wearer. Conventional neoprene braces often cause an adverse allergic reaction resulting in skin irritation. These braces are also known to be excessively abrasive where they contact the skin and are likewise known to migrate from their desired positions during activity, The combination of their abrasive character and the migration frequently results in skin irritation and laceration. Perspiration coupled with a lack of circulation further allows irritation and maceration of the skin under the cast, device, or brace, which may result in the growth of bacteria and skin erosion. If untreated, this condition can lead to infection.

15. Additionally, while the fiberglass cast shell may be impervious to water, the cotton and cloth liner is not. It holds water, dries slowly, and retains moisture against the skin. It is also weakened and tends to clump and bunch up when wetted. The accompanying lack of air to dry the skin could also encourage maceration under the cast and opening it to attack by bacteria.

16. Furthermore, unpleasant odor may develop due to retention of body oils and perspiration inside the cast. Ability to immerse or irrigate the interior of the cast without damage can be valuable in helping to prevent this.

17. Skin erosion and subsequently formed pustules that emanate from blocked hair follicles can cause extreme discomfort under a conventional cast. Additionally, urine soaked cast linings, particularly in the case of casts worn by infants, have wet padding in constant contact with the skin, resulting in ulcerations.

18. Previously taught technologies do not overcome the problem of retained dampness once liquid gets inside the lining and reaches the user's skin. None of the protective liner systems do, nor should, provide a water tight seal against immersion. Additionally, in conditions of immersion, the previously taught and otherwise valuable quality of being water impermeable while water vapor permeable becomes moot because liquid water will inevitably enter the interior of the liner by bypassing the edges of the protective materials. The problem to be addressed then becomes one of quickly getting the skin dry again.

19. Without the ability to efficiently dry the interior of orthopedic or orthotic devices, particularly casts, immersing, cleansing or rinsing the interiors becomes unfeasible.

### BRIEF SUMMARY OF THE INVENTION

1. A primary object of the invention is that it may be immersed in water without ill effect.

2. Another object of the invention is that it will quickly shed water.

3. Another object of the invention is that it be non-absorbent, resisting water vapor, liquid water, and odors.

4. Yet another object of the invention is to promote quick drying of dampness or perspiration in the interior of an orthopedic cast or other orthopedic device.

5. Another object of the invention is to provide superior, resilient protection from chaffing and/or pressure points in casts and other orthopedic devices.

6. A further object of the invention is to allow circulation of air inside and throughout the orthopedic device.

7. Still, yet another object of the invention is that it have superior strength and resilience over cotton liners that are presently the dominant lining and cushioning material of choice.

8. Another object of the invention is that it stretch and conform to the topography of the surface it covers.

9. Another object of the invention is that it resist migration and retain position as applied.

10. Another object of the invention is that it protect against contact with any cast-saw used in removing orthopedic casts.

11. A further object of the invention is to allow reuse as wrapping under compression dressings in the event of re-wrapping or change of said dressings.

12. Other objects and advantages of the present invention will become apparent from the following descriptions, taken in connection with the accompanying drawings, wherein, by way of illustration and example, an embodiment of the present invention is disclosed.

13. In accordance with a preferred embodiment of the invention, there is disclosed a machine comprised of hydrophobic synthetic polymers configured in strip(s), swath(s), tube(s), sleeve(s), glove(s), or other forms compatible with use for cushioning and/or protecting a human, animal, or plant subject from chaffing, pressure point injury, damage or discomfort from an orthopedic or non-orthopedic, cast, compression dressing, and/or brace, and to protect against a cast saw blade.

14. Also in accordance with a preferred embodiment of the invention, there is disclosed a process to cushion and/or protect a human, animal, or plant subject from chaffing, pressure point injury, damage or discomfort from an orthopedic or non-orthopedic, cast, compression dressing, and/or brace, and to protect against a cast saw blade.

### BRIEF DESCRIPTION OF THE DRAWINGS

1. The drawings constitute a part of this specification and include exemplary embodiments of the invention, which may be embodied in various forms. It is to be understood that in some instances various aspects of the invention may be shown exaggerated or enlarged to facilitate an understanding of the invention.

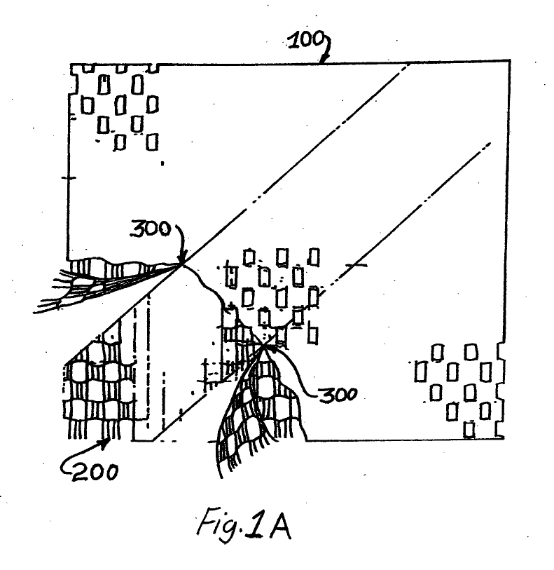

2. FIG. 1A is a drawing of the device fabric (10) used in the preferred mode with some of the scrim (20) exposed. It is cut on the bias (30) into strip form, also in accordance with the preferred mode.

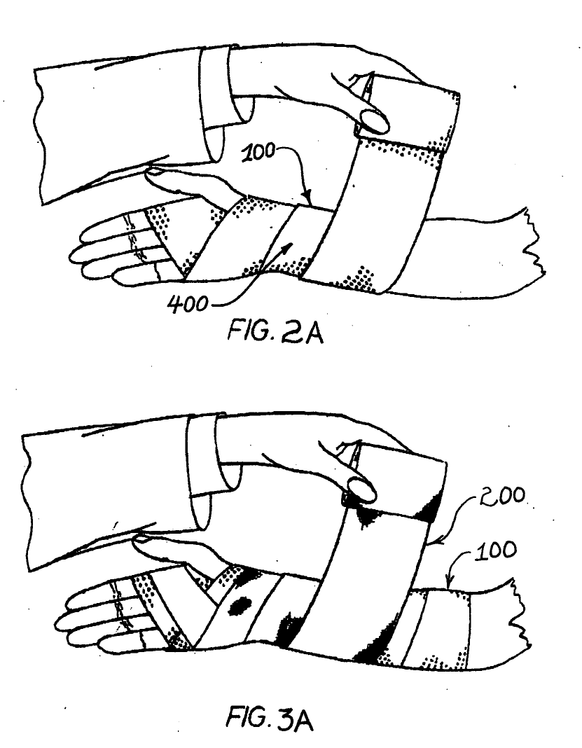

3. FIG. 2A is a drawing of the device (100) in preferred mode being applied to a hand and forearm.

4. FIG. 3A is a drawing of the device (100) in preferred mode applied to a hand and forearm and having a fiberglass cast shell (200) in the process of being wrapped over it, and held in place by adhesive (300) on the surface of the cushion material facing away from the skin.

5. FIG. 4A is a drawing of the device (100) in preferred mode in initial stage of being applied to a foot and lower leg.

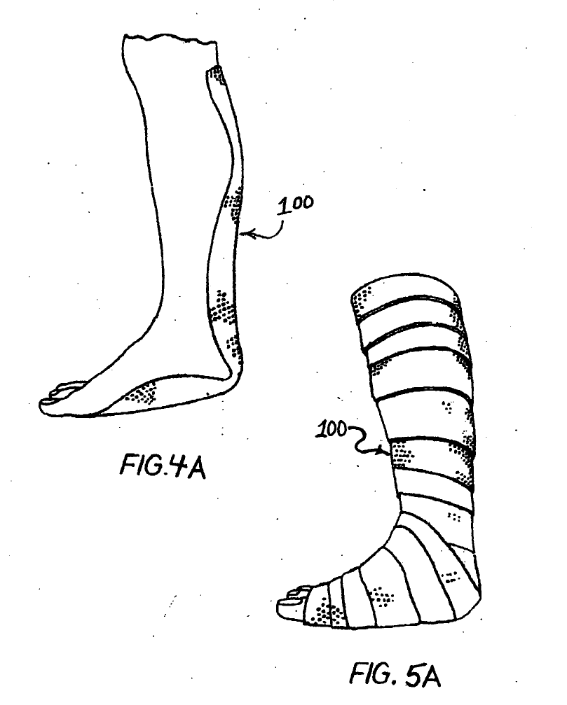

6. FIG. 5A is a drawing of the device (100) in preferred mode as applied to a foot and lower leg and ready to receive a cast shell.

### DETAILED DESCRIPTION OF THE PREFERRED EMBODIMENTS

1. Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

2. In the preferred embodiment, the present device uses materials comprised of hydrophobic synthetic polymers, in example, PTFE or Polyvinylchloride (PVC) in closed cell foam mode, for cushioning and/or protecting a subject from chaffing, pressure point Injury, damage or discomfort from an orthopedic or non-orthopedic protective device such as a cast, compression dressing, or brace, and to protect against a cast saw blade in the removal process.

3. In the preferred embodiment, the hydrophobic synthetic polymers are composed about a grid core or scrim, (See FIG. 1A, item 20) giving resilience, flexibility, and elasticity. The material is configured with numerous perforations, holes, or openings through it, and prepared before application by cutting it, on the bias, into strips, which is to say cutting along a line not parallel to the true or apparent warp, woof, texture, or grid lines of the core grid or scrim. This gives it significantly improved flexibility and elasticity to conform to the topography of the part of the body it protects and improves it's resistance to migration while in use.

4. This embodiment promotes quick-drying of dampness and/or perspiration in the interior of a protective device, orthopedic cast (FIG. 3A, item 200), compression dressing or other orthopedic or non-orthopedic device and permits improved ventilation and/or air flow within and through the interior of the protective device. It, therefore, renders practical, the complete, unprotected liquid immersion, while in use, of a orthopedic cast, compression dressing, and/or brace.

5. Its resilience and non-absorbent properties render it practical for re-use on the same subject after adjustment and/or change of the protective device. Referring to FIG. 2A, the part to be casted is placed in a position appropriate to maintain good support or fracture reduction as needed.

6. In the example Illustrated, a hand and forearm, are prepared with padding prior to application of a cast. A length of the foam padding [(100)] is wrapped around the palm between the thumb and index finger and then around the base of the thumb.

7. The material is then cut and the end taped to itself. Alternatively, the material may be held in place in whole or in part, by adhesive, alone, on the surface of the material away from the skin (see FIG. 2, item 30).

8. Additional padding is then wound up the forearm in a spiral fashion with about 20% to 30% overlap, and then cut and taped to itself near the elbow. Again, in alternative, the material may be held in place, in whole or in part, by adhesive alone on the surface of the material away from the skin (see FIG. 2, item 30).

9. Referring to FIG. 3A, a cast (200) is then applied by wrapping fiberglass around the hand and forearm in a fashion conforming to previously standardized methods. Redundant interior padding is then folded back at each end and held under the fiberglass wrap.

10. Referring to FIG. 4A, for application to a foot and ankle, a length of the material composed and prepared as above is run up the back of the calf from toes to knee. Additional wrap is placed around the foot and calf from the toes to knee with 20% to 30% overlap. In FIG. 5A, the device is ready for fiberglass casting material to be wrapped over the padding and redundant padding is folded back and included in the cast at each end.

11. When used as padding under compression dressings, the padding material needs not be discarded upon adjustment or reapplication of the dressing, but may be adjusted and use continued in place.

12. In alternative form, the bias cut material may be joined at the edges in approximately straight lines to form a tube or sleeve. In more detailed configuration it may be formed into a glove, sock, cap or other piece of apparel.

13. While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

1. A machine consisting of cushion material composed essentially of hydrophobic synthetic polymers or essentially of breathable polytetraflouroethylene, configured in strip(s), swath(s), tube(s), sleeve(s), glove(s), or other forms as a sole means of padding, and/or protecting a human, animal, or plant subject from chaffing, pressure point injury, damage or discomfort from a protective device, orthopedic cast, compression dressing, and/or other orthopedic or non-orthopedic device.

2. A machine as in claim 1, wherein the cushion material is composed about a grid core or scrim;

3. A machine as in claim 1, wherein the cushion material is configured with numerous perforations, holes, or openings;

4. A machine as in claim 1, wherein the cushion material is prepared and/or shaped by cutting and/or joining it on the bias, which is to say along a line not parallel to the true or apparent warp, woof, texture, or grid lines of the core grid or scrim;

5. A process wherein the machine as in claim 1 is employed s a means to pad, cushion and/or protect a human, animal, or plant subject from a cast saw blade in an orthopedic device removal;

6. A process wherein the machine of claim 1 is employed as a means for lining a protective device, orthopedic cast, compression dressing, and/or other orthopedic or non-orthopedic device;

7. A process wherein the machine of claim 1 is employed as a means to promote quick-drying of dampness and/or perspiration in the interior of a protective device, orthopedic cast, compression dressing or other orthopedic or non-orthopedic device;

8. A process wherein the machine of claim 1 is employed as a means to permit unprotected liquid immersion of a body part while in a protective device, such as an orthopedic cast, compression dressing, and/or other orthopedic or non-orthopedic device;

9. A process wherein the machine of claim 1 is employed as a means to permit improved ventilation and/or air flow within and through the interior of the protective device, orthopedic cast, compression dressing, and/or other orthopedic or non-orthopedic device.

10. A process wherein the machine of claim 1 is employed as a means for it's own reuse after adjustment and/or changing of the protective device, orthopedic cast, compression dressing, and/or other orthopedic or non-orthopedic device; and

11. An machine comprising an immobilizing orthopedic cast consisting of:
(a) a machine consisting of protective/cushion material composed essentially of hydrophobic synthetic polymers or essentially of breathable polytetraflouroethylene, configured in strip(s), swath(s), tube(s), sleeve(s), glove(s), or other forms as a means of padding, and/or protecting a human, animal, or plant subject from chaffing, pressure point injury, damage or discomfort from a protective device, orthopedic cast, compression dressing, and/or other orthopedic or non-orthopedic device, and to protect against a cast saw blade in the removal process; and
(b) an outer immobilizing layer of water impervious material positioned around said machine.

12. A machine as in claims 1 or 11 wherein adhesive is applied to said cushion material.

13. A machine as in claims 1 or 11 wherein said cushion material includes at least one surface coated with adhesive.

14. A process as in claim 5, 6, 7, 8, 9 or 10 wherein said cushion material of claim 1 or 11 is held in place at least in part by adhesive applied to, and present on at least one surface of, the cushion material.
